(19) United States
(12) Patent Application Publication
(10) Pub. No.: US 2010/0249810 A1
(43) Pub. Date: Sep. 30, 2010

(54) SUTURE MANAGEMENT SYSTEM FOR SURGICAL PORTAL APPARATUS INCLUDING SLOTTED RING

(75) Inventor: Eric Taylor, East Hampton, CT (US)

Correspondence Address: Tyco Healthcare Group LP;d/b/a Covidien, 555 Long Wharf Drive, Mail Stop 8-N1, Legal Department, New Haven, CT, 06511, US

(73) Assignee: Tyco Healthcare Group LP

(21) Appl. No.: 12/709,593

(22) Filed: Feb. 22, 2010

Related U.S. Application Data
(60) Provisional application No. 61/162,756, filed on Mar. 24, 2009.

(51) Int. Cl. A61B 17/04

(52) U.S. Cl. 606/148

(57) ABSTRACT

A surgical portal apparatus for use in surgical procedures incorporating at least one suture is provided. The surgical portal apparatus includes a portal member defining a longitudinal axis and having a longitudinal opening therethrough for receiving a surgical object, and a suture retaining insert configured to be selectively received in the portal member. The suture retaining insert may include a base and a plurality of spaced projections extending radially outward from the base, the projections being radially spaced whereby adjacent projections define a suture receiving passage adapted to accommodate a suture.

### CROSS REFERENCE TO RELATED APPLICATION

The present application claims the benefit of and priority to U.S. Provisional Application Ser. No. 61/162,756 filed on Mar. 24, 2009, the entire contents of which are incorporated herein by reference.

### BACKGROUND

1. Technical Field

The present disclosure relates to trocars and other surgical portal apparatus, and more particularly, relates to a suture management system for surgical portal apparatus that includes a slotted ring.

2. Background of Related Art

Trocars and other surgical portal apparatus are known, as are myriad procedures that may be preformed using such assemblies. Many of the minimally invasive procedures performed through access assemblies necessitate or are simplified by the use of one or more sutures passing through the surgical portal apparatus. Sutures extending into a body cavity through a surgical portal apparatus may be used to, for example, temporarily retain tissue, manipulate tissue, anchor tissue or operate peripheral devices. In an attempt to reduce the number of incision sites required to complete a given surgical procedure, a single surgical portal apparatus may be used to pass one or more sutures into a body cavity, in addition to providing access for one or more devices. A single anchor device may have numerous suture ends that extend therefrom and through the surgical portal apparatus. The sutures extending through the surgical portal apparatus may become tangled as each is manipulated or as one or more instruments are inserted and withdrawn from the assembly. Also, a surgeon may confuse the suture ends during the course of a surgery. Tangling or confusion of the suture ends may unnecessarily complicate the procedure and increase time necessary to complete the procedure.

Therefore, it would be beneficial to have a suture management system for use with surgical portal apparatus.

### SUMMARY

Accordingly, a surgical portal apparatus for use in surgical procedures incorporating at least one suture is provided. The surgical portal apparatus includes a portal member defining a longitudinal axis and having a longitudinal opening therethrough for receiving a surgical object, and a suture retaining insert configured to be selectively received in the portal member. The suture retaining insert may include a base and a plurality of spaced projections extending radially outward from the base. The projections are radially spaced whereby adjacent projections define a suture receiving passage adapted to accommodate a suture.

The suture retaining insert may include at least three projections. The at least three projections are arranged to define at least two suture receiving slots between adjacent projections. The portal member may include an internal shelf adjacent the internal opening for supporting the suture retaining insert. The base of the insert may include an opening for receiving a surgical object therethrough. The base may be generally annular in configuration. The surgical portal apparatus may further include a seal member disposed along the longitudinal opening of the portal member for forming a substantial seal about the surgical object.

In another aspect, the surgical portal apparatus includes a portal member defining a longitudinal axis and having a longitudinal opening therethrough for receiving a surgical object and proximal and distal ends and a plurality of individual suture retaining members positioned adjacent the proximal end of the portal member. The suture retaining members each include inner portions defining a slot for receiving a suture extending though the longitudinal opening in the portal member. The suture retaining members may each be generally C-shaped.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective side view of a surgical portal apparatus according to an embodiment of the present disclosure;

FIG. 2 is a top view of the surgical portal apparatus of FIG. 1;

FIG. 3 is a top view of an alternate embodiment of a surgical portal apparatus according to the present disclosure;

FIG. 4A is a top view of an insert according to an alternate embodiment of the present disclosure;

FIG. 4B is a side views of the insert of FIG. 4A;

FIG. 5A is a top view of an insert according to another embodiment of the present disclosure;

FIG. 5B is a side view of the insert of FIG. 5A;

FIG. 6 is a top view of a surgical portal apparatus according to another embodiment of the present disclosure;

FIG. 7A is a top view of a suture retainer of FIG. 6;

FIG. 7B is a side view of the suture retainer of FIG. 7A; and

FIG. 8 is a top view of a surgical portal apparatus according to an alternate embodiment of the present disclosure.

### DETAILED DESCRIPTION

The suture management apparatus and systems herein disclosed may be configured for use in various surgical procedures, including laparoscopic, endoscopic, arthroscopic and orthopedic surgery. The suture management systems provides passage between a subject's body cavity and the outside atmosphere and is capable of receiving surgical instruments of various sizes and configurations. Embodiments of the presently disclosed suture management systems are configured to receive, for example, clip appliers, graspers, dissectors, retractors, staplers, laser probes, photographic devices, endoscopes and laparoscopes, tubes, and the like. Such instruments are collectively referred to herein as “instruments” or “instrumentation.”

In addition to the instruments, the suture management systems also allows the passage of one or more sutures therethrough, e.g., during an arthroscopic procedure. When several sutures are introduced into the subject's body through the suture management system, the sutures might tangle with each other or be confused by a surgeon. Suture tangle and/or confusion may, at the very least, inconvenience the clinicians conducting the surgical procedure. To minimize the possibility of sutures tangling with one another or a surgeon from confusing the sutures, the suture management system incorporates a suture retaining member for retaining the one or more sutures.

Referring now to the drawings wherein like reference numerals illustrate similar components throughout the several views, there are illustrated embodiments of surgical portal apparatus according the principles of the present disclosure. As shown in the drawings and as described throughout the following description, as is traditional when referring to relative positioning on an object, the term “proximal” refers to the end of the apparatus which is closer to the user and the term “distal” refers to the end of the apparatus which is further from the user.

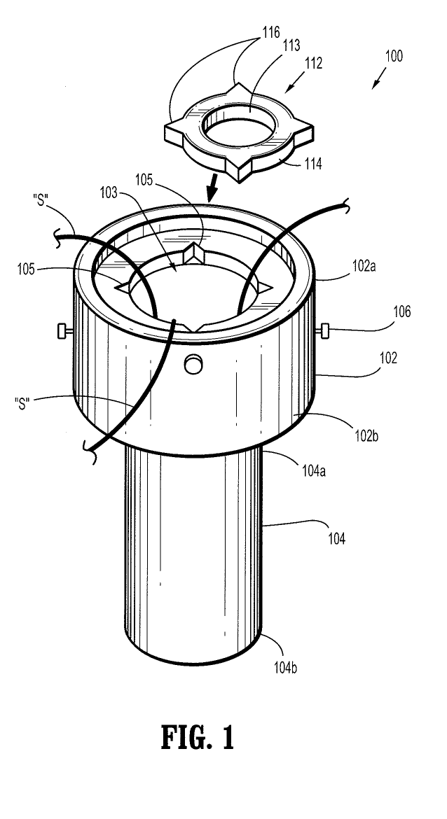

Referring initially to FIG. 1, an embodiment of a surgical portal apparatus according to the present disclosure is shown generally as surgical portal apparatus 100. Surgical portal apparatus 100 includes a portal member 102, a sleeve 104 extending distally from portal member 102, and a suture management apparatus 110 configured to engage portal member 102. Surgical portal apparatus 100 may be configured for use with any known endoscopic or laparoscopic instrument.

Portal member 102 defines a substantially cylindrical member having an open proximal end 102a, a substantially open distal end 102b and defining a passage 103 therebetween. Distal end 102b of portal member 102 may be integrally formed with sleeve 104. Alternatively, portal member 102 may be configured for selectable engagement with sleeve 104. Portal member 102 may be constructed of plastic, polymer or other like material. Portal member 102 may be disposable, or in the alternative, reusable. Portal member 102 may be rigid, or alternatively, substantially flexible. Portal member 102 may include one or more anchors 106 or other suture securing means for securing one or more sutures 10 extending through surgical portal apparatus 100. Portal member 102 may further include one or more seal members (not shown) having any seal arrangement for receiving an instrument in a sealed manner. As will be discussed in further detail below, proximal end 102a of portal member 102 is configured to receive suture management apparatus 110.

Sleeve 104 is configured to be inserted through the skin into a body cavity with the aid of an obturator (not shown), or may instead, include a blade or piercing tip for penetrating through the skin and into a body cavity. Sleeve 104 forms a substantially tubular member having proximal and distal ends 104a, 104b and defining a first longitudinal passage 103 extending therebetween. Sleeve 104 may be composed of plastic, metal, polymers or the like. Sleeve 104 may be disposable, or in the alternative, reusable. Sleeve 104 may be rigid, or alternatively, sleeve 104 may be flexible. Sleeve 104 may be open, or instead, may be configured to include one or more seal members (not shown) along the length thereof. In an alternate embodiment, proximal end 104a of sleeve 110 may be configured for operable engagement with suture management device 110.

Suture management apparatus 110 defines a suture retaining insert 112 configured for operable engagement with portal member 102. Insert 112 may be composed of plastic, polymer, metal or any other suitable material. Insert 112 includes a substantially annular base 114 and a plurality of projections or partitions 116 radial spaced thereabout. Annular base 114 defines an opening 113 configured for receipt of elongated objects therethrough. Opening 113 may be of varying diameters and may include a seal member (not shown) for receiving an instrument in a sealed manner. Projections 116 may be integrally formed with base 112. Alternatively, projections 116 may be securely affixed to and/or selectively removable from base 114. As shown, projections 116 include substantially triangular-shaped members; however, alternate configurations are envisioned.

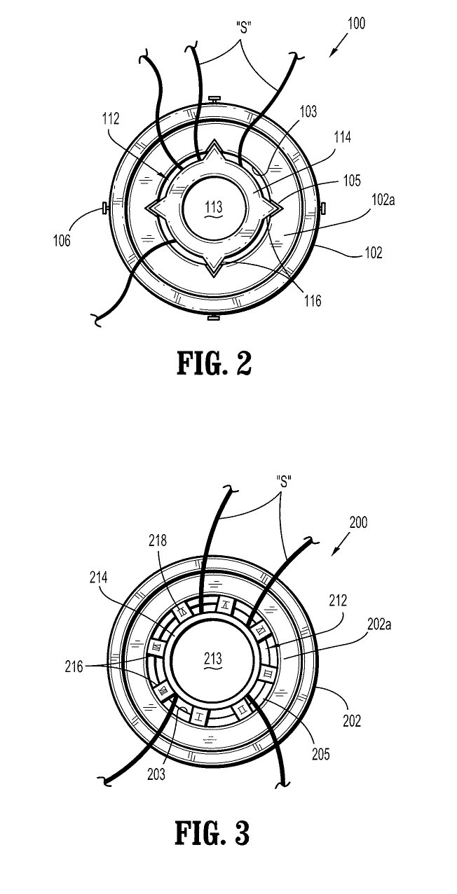

With reference still to FIGS. 1 and 2, portal member 102 includes a plurality of cut-outs 105 configured to receive insert 112. Cut-outs 105 are formed in proximal end 102a of portal member 102 about passage 103. Cut-outs 105 correspond in number and placement to projections 116 of insert 112. Cut-outs 105 are configured to receive projections 116 of insert 112 as insert 112 is received in proximal end 102 of portal member 102. As shown, cut-outs 105 are configured to loosely receive projections 116; however, cut-outs 105 and/or projections 116 may be configured such that insert 112 frictionally engages proximal end 102a of portal member 102. In an alternate embodiment, cut-outs 105 may be configured to selectively receive projections 116 in a locking manner. For example, one or more of cut-outs 105 may include a lip (not shown) configured such that rotation of insert 112 once projections 116 are received in cut-outs 105 would cause a portion of projection 116 to engage the lip, thereby securing insert 112 within proximal end 102a of portal member 102. Rotation of insert 112 in an opposite direction would cause projections 116 to disengage the lip, thereby releasing insert 112 from portal member 102.

In operation, surgical portal apparatus 100 operates in a manner similar to conventional access assemblies. Sleeve 104 is inserted through tissue, either with a piercing tip (not shown) or with the aid of an obturator (not shown). Once received through the tissue, surgical portal apparatus 100 may receive instruments through passage 103 in the absence of insert 112, or instead, through opening 113 formed in insert 112. Prior to receipt of one or more sutures “S” through surgical portal apparatus 100, insert 112 is removed from passage 103. One or more sutures “S” are then received through passage 103 of surgical portal apparatus 100 in any conventional manner. Sutures “S” are then extended radially outward from passage 103 over proximal end 102a of portal member 102 between cut-outs 105 (FIG. 1). A surgeon may then place insert 112 within passage 103, thereby retaining sutures “S” in an outer perimeter of passage 103 closer to portal member 102 (FIG. 2). Once insert 112 is in place, elongated objects may be passed through opening 113 without causing the tangling of sutures “S”. Insert 112 may be removed as necessary to add, remove and/or relocate sutures “S” within passage 103.

Turning to FIG. 3, an alternate embodiment of a suture management system of the present disclosure is shown generally as surgical portal apparatus 200. Surgical portal apparatus 200 is substantially similar in form and function to surgical portal apparatus 100 described hereinabove, and therefore will only be described as relates to the differences therebetween. Surgical portal apparatus 200 includes a portal member 202 and an insert 212. Insert 212 includes a plurality of projections 216 extending radially from annular base 214. Annular base 214 defines an opening 213 for receipt of an elongated object. Projections 216 include markings 218 to assist in identifying one or more sutures “S” retained between projections 216. Markings 218 may be letters, numbers, symbols, colors or other identifying feature. Alternatively, or in addition, markings (not shown) may be included on proximal end 202a of portal member 202. Insert 212 is configured to be received within a proximal end 202a of portal member 202. Proximal end 202a includes a recessed portion or shelf 205. Shelf 205 is configured to engaging projections 216 of insert 212 as insert 212 is received within passage 203. The configuration of insert 212 and portal member 202 permits insert 212 to be rotated within passage 203 while maintaining one or more sutures “S” between projections 216 and away from opening 213. In this manner, insert 212 operates to prevent sutures “S” from becoming tangled and/or confused. Insert 212 further permits selective relocation of sutures “S” about passage 103 without removing insert 212 from portal member 202.

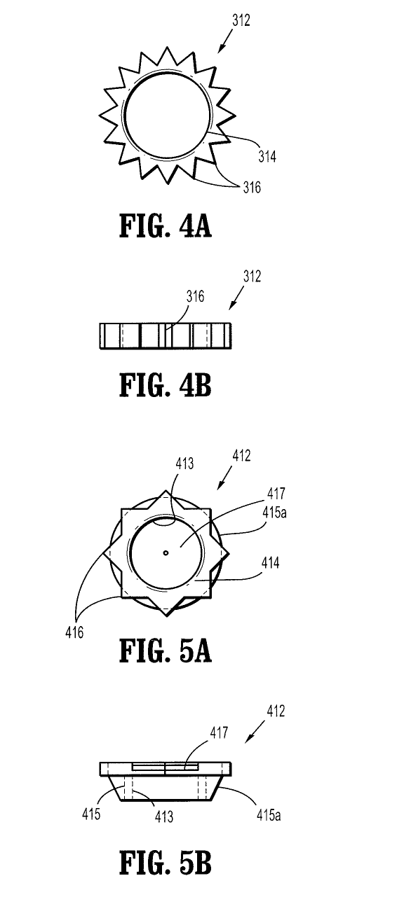

With reference now to FIGS. 4A and 4B, an alternate embodiment of a suture retaining insert according to the present disclosure is shown generally as suture retainer insert 312. Insert 312 is substantially similar in form and function to inserts 112, 212 described hereinabove. Insert 312 includes a plurality of projections 316 extending from an annular base 314. Projections 316 are configured to retain a single suture “S” (FIG. 1) therebetween. In this manner, sutures “S” may be maintained completely separate from one another. Portal members 102 (FIGS. 2) and 202 (FIG. 3) may be configured to receive insert 312.

Referring now to FIGS. 5A and 5B, another embodiment of a suture retaining insert according to the present disclosure is shown generally as suture retaining insert 412. Insert 412 is substantially similar to inserts 112, 212, 312 described hereinabove. Insert 412 includes a plurality of projections 416 extending from an annular base 414. Annular base 414 defines an opening 413 configured for receiving an elongate object therethrough. Insert 412 further includes a distal extension 415 extending from base 414 and a seal member formed in opening 413. Distal extension 415 is sized and dimensioned to be received within passage 103, 203 of access assemblies 100, 200 such that projections 416 engage cut-outs 105 or shelf 205, respectively. A tapered deformable coating 415a extends about distal extension 415. Deformable coating 415a is configured to frictionally engage proximal end 102a, 202a of portal member 102, 202, respectively, as insert 412 is received within passage 103, 203, respectively, thereby securely retaining suture “S” within passage 103, 203 against portal member 102, 202. Furthermore, deformable coating 415a creates a seal between portal member 102, 202 and insert 412 thereby preventing escape of insufflation gas through surgical portal apparatus 100, 200, respectively. Seal member 417 permits an instrument to be received through insert 412 in a sealed manner.

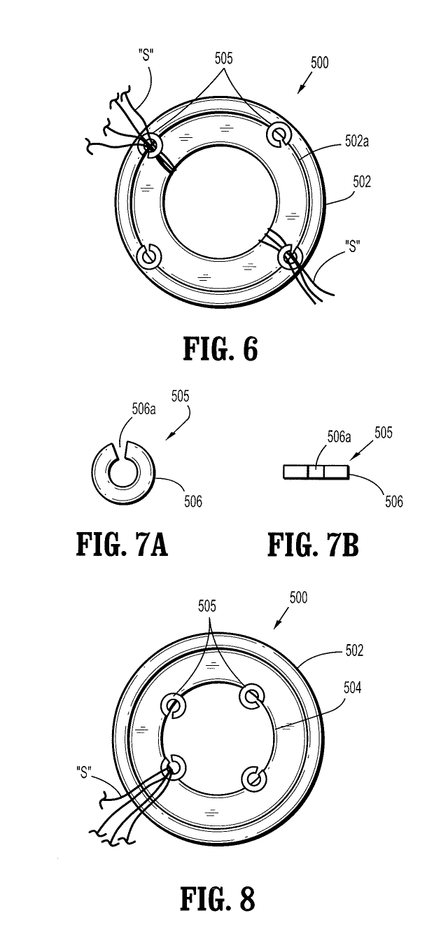

Turning now to FIG. 6, an alternate embodiment of a surgical portal apparatus according to the present disclosure is shown generally as surgical portal apparatus or surgical portal apparatus 500. Surgical portal apparatus includes a portal member 502 defining a passage 503 configured for receipt of an elongated object. Positioned about a proximal end 502a of portal member 502 is a plurality of suture retainers 505. With particular reference to FIGS. 7A and 7B, suture retainers include a substantially flat annular base 506 including a slot 506a for receiving one or more sutures “S”. Suture retainers 505 maintain suture “S” away from passage 503, thereby preventing an instrument (not shown) inserted through surgical portal apparatus 500 from engaging and causing the entangling of sutures “S”. Suture retainers 505 may be integrally formed with, securely affixed to and/or removable attached to portal member 502. In an alternate embodiment (FIG. 8), suture retainers are positioned on a proximal end of sleeve 504.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

1. A surgical portal apparatus for use in surgical procedures incorporating at least one suture, which comprises:
a portal member defining a longitudinal axis and having a longitudinal opening therethrough for receiving a surgical object; and
a suture retaining insert configured to be selectively received in the portal member, the suture retaining insert including a base and a plurality of spaced projections extending radially outward from the base, the projections being radially spaced whereby adjacent projections define a suture receiving passage adapted to accommodate a suture.

2. The surgical portal apparatus according to claim 1, wherein the suture retaining insert includes at least three projections, the at least three projections being arranged to define at least two suture receiving slots between adjacent projections.

5. The surgical portal apparatus of claim 1, wherein the portal member includes an internal shelf adjacent the internal opening for supporting the suture retaining insert.

6. The surgical portal apparatus of claim 1, wherein the annular base of the insert includes an opening for receiving a surgical object therethrough.

7. The surgical portal apparatus of claim 1, wherein the base is generally annular in configuration.

8. The surgical portal apparatus of claim 1, further including a seal member disposed along the longitudinal opening of the portal member for forming a substantial seal about the surgical object.

9. A surgical portal apparatus, which comprises:
a portal member defining a longitudinal axis and having a longitudinal opening therethrough for receiving a surgical object and proximal and distal ends; and
a plurality of individual suture retaining members positioned adjacent the proximal end of the portal member, the suture retaining members each including inner portions defining a slot for receiving a suture extending though the longitudinal opening in the portal member.

10. The surgical portal apparatus of claim 9, wherein the suture retaining members are each generally C-shaped.
